(19) United States
(12) Patent Application Publication
(10) Pub. No.: US 2026/0115214 A1
(43) Pub. Date: Apr. 30, 2026

(54) APPLICATION OF INHIBITORS OF GLYCOLYTIC PATHWAY IN PREPARATION OF DRUGS FOR PREVENTING AND TREATING PORCINE REPRODUCTIVE AND RESPIRATORY SYNDROME VIRUS (PRRSV) INFECTION

(71) Applicant: NANJING AGRICULTURAL UNIVERSITY, Nanjing, Jiangsu (CN)

(72) Inventors: Ping JIANG, Nanjing (CN); Xuewei LIU, Nanjing (CN); Juan BAI, Nanjing (CN); Xianwei WANG, Nanjing (CN)

(73) Assignee: NANJING AGRICULTURAL UNIVERSITY, Nanjing, Jiangsu (CN)

(21) Appl. No.: 17/766,825

(22) PCT Filed: Jul. 14, 2020
(86) PCT No.: PCT/CN2020/101896

§ 371 Date: Apr. 29, 2025

(30) Foreign Application Priority Data
Jul. 2, 2020 (CN) 202010628194.5

(51) Int. Cl. A61K 31/7004; A61K 31/198; A61K 31/366; A61P 31/14

(52) U.S. Cl. CPC A61K 31/7004; A61K 31/198; A61K 31/366; A61P 31/14

(57) ABSTRACT

The present invention discloses an application of inhibitors of glycolytic pathway in preparing drugs for preventing and treating porcine reproductive and respiratory syndrome virus (PRRSV) infection. The present invention found that the inhibitors of glycolytic pathway can be used to prevent and treat PRRSV infection, especially, oral administration of the lactate dehydrogenase inhibitor, sodium oxamate or Galloflavin, can significantly inhibit PRRSV replication in pigs, and the present invention found a new use of the inhibitors of glycolytic pathway, especially the lactate dehydrogenase inhibitor (sodium oxamate or Galloflavin), as a PRRSV antagonist for the first time. The sodium oxamate and Galloflavin have clear anti-PRRSV medicinal ingredients, are quality controllable, safe and non-toxic, and have a good application prospect in prevention and treatment of PRRSV.

### TECHNICAL FIELD

The present invention belongs to the technical field of veterinary prevention and treatment drugs, and in particular relates to an application of inhibitors of glycolytic pathway in preparation of drugs for preventing and treating porcine reproductive and respiratory syndrome virus (PRRSV) infection.

### BACKGROUND

The causative agent of porcine reproductive and respiratory syndrome (PRRS) is porcine reproductive and respiratory syndrome virus (PRRSV), belonging to the family Arteriviridae and being a positive stranded ssRNA virus. The virus infection can cause reproductive disorders in pregnant sows, respiratory disorders in pigs of all ages, immune suppression in pigs, and secondary infection-promoting diseases, causing huge economic losses to domestic and foreign pig breeding industry. Commercial vaccines against PRRSV are not yet ideal. The cross-protection ability of vaccines against strains is not complete, and the replication process of live virus in pigs has the possibilities of virulence enhancement and recombination with wild viruses to form new strains. Therefore, although a PRRSV vaccine can provide certain protection to piglets, the self-diversity caused by characteristics including recombination and deletion of PRRSV makes it evade an immune surveillance effect of the vaccine and become a huge challenge for prevention and treatment of PRRS. Drugs for antagonizing PRRSV infection are an important research direction.

Many host antiviral factors, such as interferon-stimulated genes (ISGs), Viperin, Mx2, 2′,5′-oligoadenylate synthase 1 (OAS1), interferon-induced tetrapeptide repeat protein 3 (IFIT3), and CH25H, have been reported to have antiviral activity against PRRSV infection. In addition, some microRNAs, siRNA and shRNA have also been proved to inhibit PRRSV replication. However, these proteins or small RNAs are easily degraded by enzymes in animals, making it difficult to perform in vivo experiments with them. Some antiviral compounds or natural compounds also have the effect of antagonizing PRRSV. However, the antiviral mechanism of these drugs and their antiviral effects in pigs have not been thoroughly studied, and because PRRSV is prone to mutation, the antiviral drugs targeting PRRSV are likely to cause drug-resistant mutations in the virus, thus making the drugs ineffective. Therefore, it is a crucial research idea to find new and effective antiviral drugs.

### SUMMARY

The objective of the present invention is to provide an application of inhibitors of glycolytic pathway in preparation of drugs for preventing and treating porcine reproductive and respiratory syndrome virus (PRRSV) infection.

### The Objective of the Present Invention May be Achieved Through the Following Technical Solutions

An application of inhibitors of glycolytic pathway in preparing drugs for preventing and treating PRRSV infection.

As a preferred technical solution, the inhibitors of glycolytic pathway are a hexokinase inhibitor and/or a lactate dehydrogenase inhibitor.

Further preferably, the hexokinase inhibitor is 2-deoxy-D-glucose (2-DG); and the lactate dehydrogenase inhibitor is at least one of sodium oxamate and Galloflavin.

The above drugs are prepared from the inhibitors of glycolytic pathway as an active ingredient.

The above drugs also contain a pharmaceutically acceptable carrier.

The dosage form of the above drugs is any one acceptable in veterinary pharmacy.

In the research of the present invention, it is first found that glyceraldehyde-3-phosphate dehydrogenase (GAPDH) in cells can promote PRRSV replication through its glycolytic enzymatic activity. It is further found that 2-deoxy-D-glucose (2-DG), an inhibitor of the key enzyme hexokinase in the first step of glycolysis, and sodium oxamate and Galloflavin, inhibitors of the key enzyme lactate dehydrogenase in the last step of glycolysis, have the ability to resist PRRSV infection in vitro. In view of this, we conducted a pig test and found that sodium oxamate and Galloflavin can significantly inhibit PRRSV replication in pigs by oral administration, and have a therapeutic effect on PRRSV-infected piglets and important application value.

### Beneficial Effects of the Present Invention are as Follows

The present invention found that the inhibitors of glycolytic pathway can be used to prevent and treat PRRSV infection, especially, oral administration of the lactate dehydrogenase inhibitor, sodium oxamate or Galloflavin, can significantly inhibit PRRSV replication in pigs, and the present invention found a new use of the inhibitors of glycolytic pathway, especially the lactate dehydrogenase inhibitors (sodium oxamate or Galloflavin), as PRRSV antagonists for the first time. The sodium oxamate and Galloflavin have clear anti-PRRSV medicinal ingredients, are quality controllable, safe and non-toxic, and have a good application prospect in prevention and treatment of PRRS.

### BRIEF DESCRIPTION OF THE DRAWINGS

FIG. 1 shows that overexpression of GAPDH promotes PRRSV replication.

FIG. 2 shows that knockdown of GAPDH inhibits PRRSV replication.

FIG. 3 shows that GAPDH promotes PRRSV replication in an enzymatic activity-dependent manner.

FIG. 4 shows that inhibitors of glycolytic pathway 2-DG reduces the level of PRRSV replication.

FIG. 5 shows that sodium oxamate reduces the level of PRRSV replication on Marc-145 cells.

FIG. 6 shows that Galloflavin reduces the level of PRRSV replication on Marc-145 cells.

FIG. 7 shows the clinical symptoms of PRRSV-infected and drug-treated piglets.

FIG. 8 shows the results of viral loads determined in the lungs of PRRSV-infected and drug-treated piglets.

FIG. 9 shows the gross pathological changes in the lungs of PRRSV-infected and drug-treated piglets.

FIG. 10 shows the histopathological changes in the lungs of PRRSV-infected and drug-treated piglets.

### DETAILED DESCRIPTION

Further descriptions are provided below with reference to specific embodiments.

### 1. Materials and Methods

### 1.1 Cells and Viruses

Highly pathogenic PRRSV strains BB0907 and Marc-145 cells are maintained in the laboratory and are conventional strains and cells disclosed in the prior art.

### 1.2 Main Reagents

Sodium oxamate was purchased from Nanjing Jin Yibai Biological Technology Co. Ltd., with a purity of >98%; 2-deoxy-D-glucose (2-DG) was purchased from Sigma; Galloflavin was purchased from APExBIO; an enhanced CCK8 kit was purchased from Beyotime Biotechnology Co., Ltd.; an anti-PRRSV N protein antibody (2H7) was prepared and preserved by graduate students of the laboratory of the inventors; a labeled Alex 594 goat-anti-mouse fluorescent secondary antibody was purchased from Beyotime Biotechnology Co., Ltd.; reverse transcriptase HiScript II 1st Strand cDNA Synthesis Kit and AceQ® qPCR SYBR® Green Master Mix were purchased from Nanjing Vazyme Biotech Co., Ltd.; dimethyl sulfoxide (DMSO) was purchased from Sigma; and other conventional reagents were of analytical grade.

Biochemical techniques used in the present invention are all conventional techniques in the field: in the following experimental procedures, unless otherwise specified, all experimental operations are conducted in accordance with parts in the following experimental manuals or literatures, including: [US] J. Sambrook et al., Molecular Cloning: A Laboratory Manual; Zhao Yongfang et al., Principle and Application of Biochemistry Technology (Second Edition); and Zhu Jian et al., Biochemistry Experiments [M].

### 1.3 Construction of Plasmid

Based on the gene sequences of monkey glyceraldehyde-3-phosphate dehydrogenase (GAPDH, GenBank: NM_001195426.1) and porcine GAPDH (pGAPDH, GenBank: AF017079.1) in the Genbank database, specific PCR primers were designed using Primer premer 5, and EcoR I and Nhe I restriction sites and protective bases were added at both ends of the primers (Table 1). The cDNAs of Marc-145 cells and porcine alveolar macrophages (PAM) were used as templates for conducting amplification using a Primer STAR high-fidelity enzyme, respectively. To construct GAPDH single- or double-amino acid mutants C152S, H179F, and C152S&H179F, primers containing GAPDH mutation sites were designed (Table 1), and GAPDH mutants were amplified by fusion PCR using the Primer STAR high-fidelity enzyme. By means of double digestion and ligation, a target fragment was cloned into a pCAGGS vector plasmid.

TABLE 1
Primer sequence of PCR

| Primer | Sequence (5′→3′) |
|---|---|
| GAPDH-HA-F | AGCGTAGTCTGGGACGTCGTATGGGTA |
| GAPDH-HA-R | TAGCTAGCTTAAGCAACGTCCGGAACGTCGTAC |
| | GGGTACTCCTTGGAGGCCATG |
| GAP-C152S- | CAGCAACGCCTCCTCCACCACCAACTGCTTAGC |
| HA-2-F | |
| GAP-C152S- | TAAGCAGTTGGTGGTGGAGGAGGCGTTGCTGAC |
| HA-1-R | |
| GAP-H179F-HA-F | ACTCATGACCACAGTCTTCGCCATCACTGCCAC |
| GAP-H179F-HA-R | GTGGCAGTGATGGCGAAGACTGTGGTCATGAGT |

### 1.4 RNA Interference

A 24-well plate was inoculated with Marc-145 cells. When the cells reached a confluence of 60%, the cells were transfected with 10 nmol and 15 nmol siGAPDH or 15 nmol siNC. After 24 hours of transfection, the cells were infected with 0.01 MOI PRRSV and incubated in a 37° C. incubator for 1 h. After the cell surface was washed, a medium was replaced with a fresh DMEM nutrient solution containing 2% FBS, and the cells were cultured at 37° C. for 36 h, and collected for conducting Western blotting. The sequences of interfering fragments are: siGAPDH: 5′-CGG GAA GCU CAC UGG CAUG-3′; siNC: 5′-UUC UCC GAA CGU GUC ACG UTT-3′.

### 1.5 Detection of GAPDH Enzymatic Activity

The enzymatic activities of GAPDH-HA and mutants thereof (C152S, H179F and C152S&H179F) were detected by a GAPDH activity detection kit (ab204732, Abcam). The method was based on conversion of NAD+ to NADH, and the enzymatic activity of the GAPDH was calculated by detecting the change of absorbance of a reaction system at 450 nm within 30 min at 37° C. See the manual for specific operation methods.

### 1.6 Determination of Cytotoxicity

Marc-145 cells were cultured in 10% FBS-DMEM containing different concentrations of sodium oxamate (1-250 mM), Galloflavin (0-2.5 mM) and 2-deoxy-D-glucose (0-20 μM), and incubated at 37° C. for 48 h. Cell viability was detected with an enhanced CCK8 kit. DMSO was used as a negative control.

### 1.7 Determination of Virus Titer

Marc-145 cells were cultured in a 96-well plate. When the cell confluence reached 70%, a nutrient solution was pipetted, and the cell surface was washed 3 times with PBS. Ten-fold serial dilutions of PRRSV were added to the cells and incubated at 37° C. for 1 h. A medium was replaced with fresh 2% FBS-DMEM. Virus titers were determined 5 days post inoculation (dpi) by endpoint dilution assay. 50% tissue culture infectious dose (TCID₅₀) was calculated by a Reed-Muench method.

### 1.8 Experimental Design of Piglet Treatment

20 5-week-old commercial piglets (purchased from a farmer in Nantong, Jiangsu) were detected by ELISA and PCR, and were all negative in antibodies and antigens of PRRSV, PCV2, PRV and CSFV. The piglets were randomly divided into 3 groups (5 in each group): the first group is a positive control group (Positive)-PRRSV infected and untreated; the second group was a sodium oxamate treatment group (+oxamate)-PRRSV infected and sodium oxamate treated; the third group was a Galloflavin treatment group (+Gal)-PRRSV infected and Galloflavin treated; and the third group was a negative control group (Mock)-uninfected and untreated. The piglets in the positive control group and the treatment groups were injected intranasally and intramuscularly with 1 ml of PRRSV BB0907 strain (3*10⁵ TCID₅₀), respectively. 24 hours after the infection, the treatment groups were orally administered with sodium oxamate (7 mg) and Galloflavin (10 μg) every day. After the infection, the piglets were monitored daily for health status, rectal temperature and body weight. Serum samples were collected on days 1, 4, 7, 10 and 14 post challenge. All piglets were killed and dissected on day 14, and serum and lung tissue were collected for viral load determination and histopathological examination.

### 1.9 Real-Time Fluorescent Quantitative PCR

1.9.1 Relative Fluorescent Quantitative Detection of Relative Level of PRRSV mRNA in Cells

Cellular RNA was extracted following operating instructions of an OMEGA RNA virus extraction kit. After reverse transcription into cDNA, RNA was extracted according to operating instructions of the OMEGA RNA virus extraction kit. Relative fluorescent quantification was conducted after reverse transcription, and primer sequences used are shown in Table 2:

TABLE 2
Primer sequences of qPCR

| Primer | Sequence (5′→3′) |
|---|---|
| mGAPDH-Fwd | 5′ CCTTCCGTGTCCCTACTGCCAA 3′ |
| mGAPDH-Rev | 5′ GACGCCTGCTTCACCACCTTCT 3′ |
| PRRSV-ORF7-Fwd | 5′ AAACCAGTCCAGAGGCAAG 3′ |
| PRRSV-ORF7-Rev | 5′ TCAGTCGCAAGAGGGAAAT 3′ |

By a 2-ΔΔCt method, the relative content of mRNA in each gene in the samples was calculated by a CT value.

### 1.9.2 Absolute Fluorescent Quantitative Detection of Content of PRRSV RNA in Lung and Serum

About 1 g of lung tissue was homogenized in 3 ml of PBS. After three freeze-thaw cycles, the tissue homogenate was centrifuged at 5000 rpm for 10 min, and the tissue homogenate supernatant was collected. The collected blood was centrifuged at 5000 rpm for 5 min and serum was collected. Nasal swabs were rinsed in 1 ml of PBS for 2 min, the PBS was centrifuged at 5000 rpm for 10 min, and the nasal swab dilution was collected. RNA was extracted according to operating instructions of an OMEGA RNA virus extraction kit. 2 μL of 5*qRTSuperMix (10 μL reaction system) was added to each 8 μL of the extracted RNA sample, and after thorough mixing, the mixture was subjected to a reverse transcription reaction on a PCR machine, and the reaction program was: 25° C. for 10 min; 50° C. for 30 min; and 85° C. for 5 min. The product was frozen at −70° C. and stored for use. Using the above cDNA as a template, the contents of PRRSV nucleic acids in the serum, the lung tissue and the nasal swab were detected by SYBR Green real-time PCR. The primer sequences were F: 5′-AATAACAACGGCAAGCAGCAG-3′; and R: 5′-CCTCTGGACTGTTTTGTTTGG-3′. The reaction system contains 10 μL of 2×Power SYBR Green PCR Master Mix (ABI company), and 2 μL of cDNA, and the concentration of primers F/R was 400 nmol/L. The reaction was conducted on an ABI 7300 real time PCR machine. The reaction program was: pre-denaturation at 95° C. for 2 min, 95° C. for 15 s, and 61° C. for 31 min for a total of 40 cycles. A standard curve was created with a recombinant plasmid containing the PRRSV ORF7 gene, and then using a CT value, the PRRSV virus contents in the lung tissue, the serum and the nasal swab were calculated according to the standard curve.

### 1.10 Clinical Symptoms and Pathological Observation

### 1.10.1 Observation and Statistics of Clinical Symptoms

The rectal temperature of the piglets was measured every day after the challenge; and clinical symptoms, including coat, skin, dietary intake, diarrhea, and mental state, were observed. The body weight of the piglets was measured before and after the challenge. A statistical method of the clinical symptoms is as follows: according to clinical manifestations (mental state, coat, and diarrhea), breathing and coughing conditions, these three indicators are scored respectively, and the score ranges from 1 to 4 points. The more severe the symptoms, the higher the score. 1 point means normal, 2 points mean mild symptoms, 3 points mean severe symptoms, and all indicators of dead pigs are 4 points. The piglets in each group were scored every day after the challenge, and the sum of scores of the three indicators was the clinical score of each pig every day. Data analysis was conducted according to the statistical results in 14 days after the challenge.

### 1.10.2 Scoring Method for Gross Lung Lesions

The score was assigned according to the proportion of each lobe: 10 points each for apical and cardiac lobes, 27.5 points for diaphragmatic lobes, 5 points for accessory lobes, and the total score was 100 points. Each part was scored according to the degree of lesions. The larger the lesion area, the higher the score. The sum of all parts was the final score of the lung. After three times of independent scoring, the lung scores of each group were statistically analyzed. All the piglets were killed and dissected 14 days after the challenge, and the pathological changes in the lungs of each piglet were observed. Edema, interstitial widening and carnification were observed and statistically analyzed.

### 1.10.3 Preparation and Observation of Pathological Sections

Tissue blocks no larger than 4 mm×2 cm² were removed from lung tissue of the pigs, fixed with 4% paraformaldehyde, dehydrated in alcohol in a gradient manner, transparentized in xylene, embedded in paraffin, trimmed, sectioned, and stained with hematoxylin and cosin. Lung sections were observed under a microscope, and the pathological scoring rules were: 0 point: no lesions; 1 point: mild, focal/multifocal interstitial pneumonia (<50%); 2 points: moderate, multifocal consolidation (50-75%); 3 points: severe, extensive patchy consolidation (75-90%); and 4 points: severe diffuse (>90%). GraphPad 7 software was used for statistical analysis of data, and differences among groups were compared. P<0.05 means significant difference (ns means P>0.05; * means P<0.05; ** means P<0.01; *** means P<0.001; and **** means P<0.0001).

### 2. Results

### 2.1 Discovery of Important Role of GAPDH Glycolytic Activity in PRRSV Replication

### 2.1.1 GAPDH Facilitates PRRSV Replication

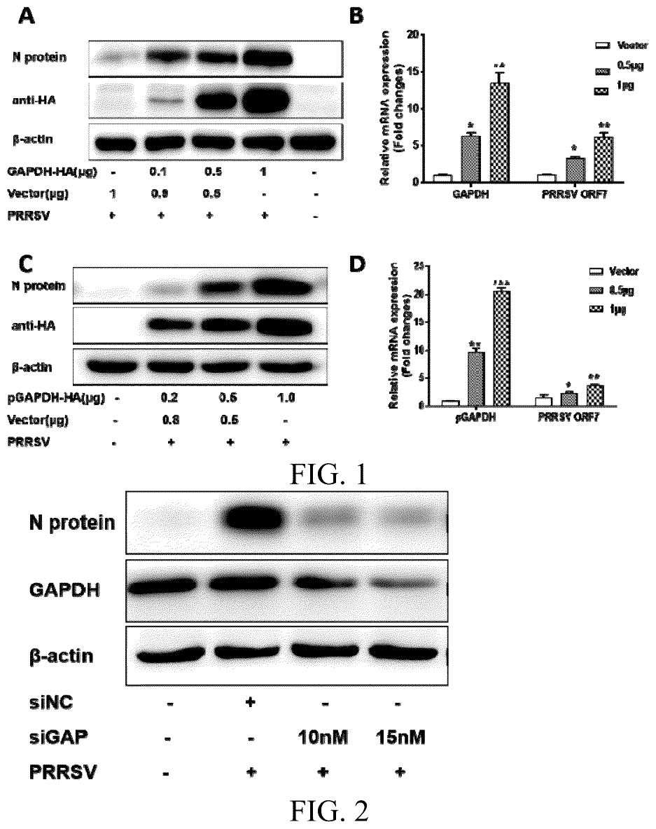

Marc-145 cells were transfected with monkey GAPDH eukaryotic expression plasmids, and inoculated with 0.1 MOI of PRRSV 24 h later. A cell lysate was collected after 36 h of infection to detect PRRSV replication. The results showed that overexpression of GAPDH significantly increased replication of PRRSV, and the expression level of PRRSV-N protein and viral RNA level gradually increased with the increase of GAPDH expression (FIG. 1A and FIG. 1B). The amino acid sequence homology between porcine GAPDH (pGAPDH) and monkey GAPDH is 97%. We measured the level of PRRSV replication in pGAPDH-transfected cells, and the results showed that porcine GAPDH (pGAPDH) and monkey GAPDH had similar effects on PRRSV replication (FIG. 1C and FIG. 1D).

To further clarify the effect of GAPDH on PRRSV replication, we designed siRNA (siGAPDH) against the GAPDH gene and transfected Marc-145 cells with the siGAPDH (at a transfection dose of 10 nM and 15 nM). An irrelevant interference control group (transfected with 15 nM of siNC) was set up, and 24 h after transfection, the irrelevant interference control group was infected with 0.1 MOI of PRRSV. After being cultured for 30 h, the cells were collected and lysed, and the relative content of PRRSV N proteins was detected by Western blotting. The results are shown in FIG. 2. Compared with cells in the siNC control group, siGAPDH could effectively reduce expression of endogenous GAPDH (P<0.01), and significantly reduce expression of PRRSV N proteins (P<0.01).

### 2.1.2 GAPDH Promotes PRRSV Replication Through Glycolytic Activity

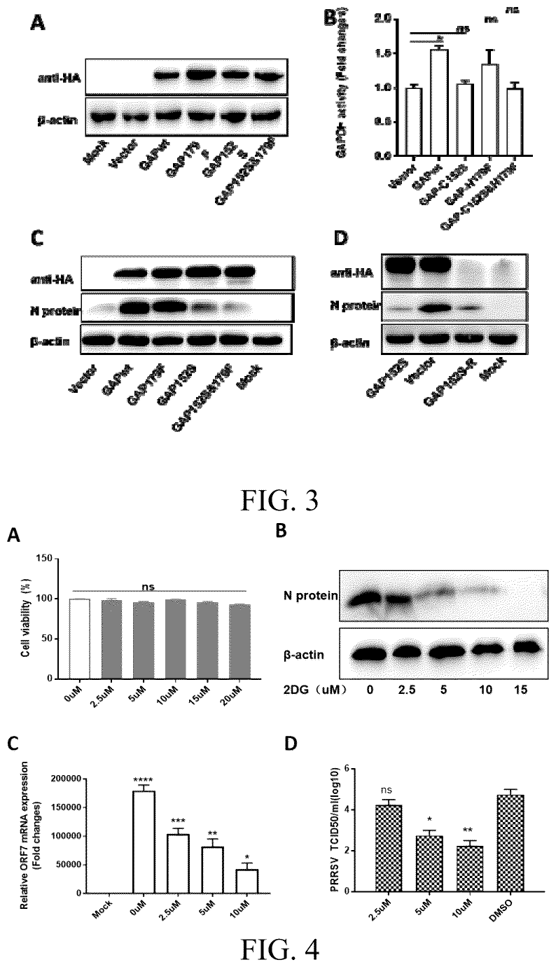

Key amino acids related to GAPDH enzymatic activity were mutated, and three GAPDH mutant recombinant plasmids were constructed: GAP-C152S, GAP-H179F and GAP-C152S&H179F. Marc-145 cells were transfected and detected by Western blotting. The protein expression levels of the three mutants were basically the same (FIG. 3A). Marc-145 cells were collected and the GAPDH enzymatic activity was determined. The results in FIG. 3B showed that compared with the wild-type GAPDH, the GAPDH enzymatic activity of the two mutants with cysteine at position 152 (Cys-152) mutated significantly decreased. Then the three mutants were transfected, infected with PRRSV, cultured for 36 h, and detected for the PRRSV-N protein level by Western blotting. The results are shown in FIG. 3C: the GAPDH with the cysteine at position 152 (Cys-152) mutated could not promote PRRSV replication. When GAP-C152S was reversibly mutated, its effect of significantly promoting PRRSV replication was restored (FIG. 3D). Based on the above results, GAPDH promotes PRRSV replication through the glycolytic activity, and the cysteine at position 152 (Cys-152) is an important active site.

### 2.2 Discovery of Significant Inhibition Effect of Inhibitors of Glycolytic Pathway on PRRSV Replication

### 2.2.1 2-DG Inhibits PRRSV in Marc145 Cells

To explore the effect of glycolysis in cells on PRRSV replication, an inhibitor of glycolytic pathway, 2-DG, was used to inhibit glycolysis in cells. First, the cytotoxicity of 2-DG on Marc-145 cells was determined by a CCK8 cytotoxicity assay, and the 2-DG was found to be almost non-toxic to the cells at a dose 20 μM or less (FIG. 4A). After the Marc-145 cells were infected with PRRSV (0.01 MOI), the Marc-145 cells were treated with 0 μM, 2.5 μM, 5 μM, 10 μM and 15 μM of 2-DG respectively. After 36 hours, the inhibition efficiency of the 2-DG on PRRSV on the Marc-145 cells was detected by Western blotting. As shown in FIG. 4B, the 2-DG had an inhibition efficiency of about 50% against PRRSV at a concentration of 5 μM. To further determine the anti-PRRSV activity of the 2-DG, viral TCID₅₀ in a cell culture medium and a PRRSV ORF7 mRNA level in the cells were detected, and the results showed that the virus titer and the ORF7 mRNA level decreased in a dose-dependent manner (FIG. 4C and FIG. 4D).

### 2.2.2 Sodium Oxamate Inhibits PRRSV in Marc-145 Cells

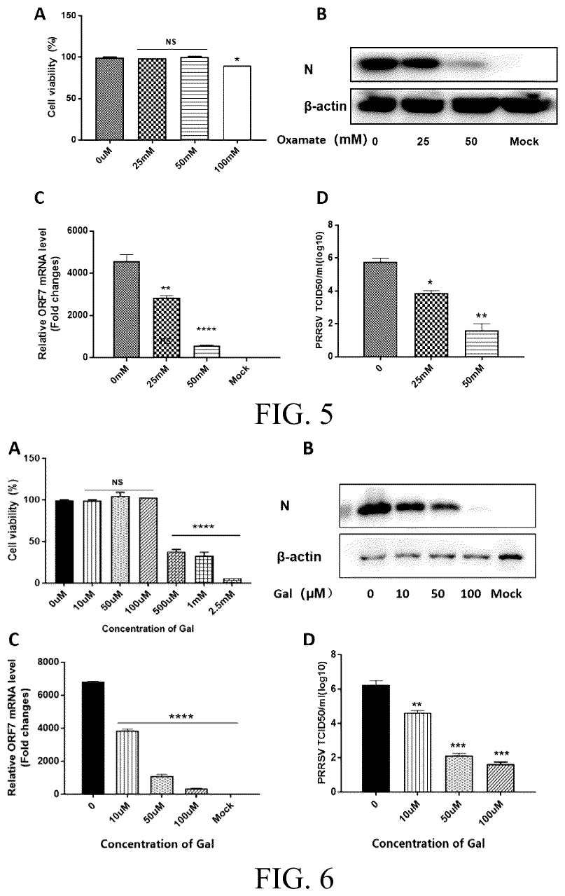

To further verify the effect of glycolysis on PRRSV, PRRSV-infected Marc-145 cells were treated with a key enzyme in the last step of glycolysis, that is, an inhibitor of lactate dehydrogenase, sodium oxamate (SO). A control group was set up to detect whether there was a difference in the level of virus replication. The cytotoxicity of SO on Marc-145 cells was first determined by a CCK8 cytotoxicity assay, and the SO was found to start to be toxic to the cells at 100 mM (FIG. 5A). After the Marc-145 cells were infected with PRRSV (0.01 MOI), the Marc-145 cells were treated with 0 mM, 25 mM and 50 mM of SO. 36 h after infection, the PRRSV N protein expression level, virus titer and PRRSV ORF7 mRNA level were detected by Western blotting, TCID₅₀ and RT-PCR respectively. The results showed that SO could reduce PRRSV replication in the cells in a dose-dependent manner (FIG. 5B-FIG. 5D).

### 2.2.3 Galloflavin Inhibits PRRSV in Marc-145 Cells

PRRSV-infected Marc-145 cells were treated with an inhibitor of lactate dehydrogenase (Galloflavin, Gal), and a control group was set up to detect differences in viral replication levels. The cytotoxicity of Gal on Marc-145 cells was first determined by a CCK8 cytotoxicity assay, and the Gal was found to start to be toxic to the cells at 500 μM (FIG. 6A). After Marc-145 cells were infected with PRRSV (0.01 MOI), the Marc-145 cells were treated with 10 μM, 50 μM and 100 μM of Gal. 36 h after infection, the expression level of PRRSV N protein was detected by Western blotting, and the results showed that Gal could decrease PRRSV replication in the cells in a dose-dependent manner (FIG. 6B). In addition, A PRRSV ORF7 mRNA level and the virus titer were detected by TCID₅₀ and RT-PCR. The results showed that Gal could decrease the mRNA level of PRRSV in a dose-dependent manner (FIG. 6C-FIG. 6D).

2.3 Sodium Oxamate and Galloflavin have Therapeutic Effects on PRRSV-Infected Piglets

### 2.3.1 Clinical Symptoms

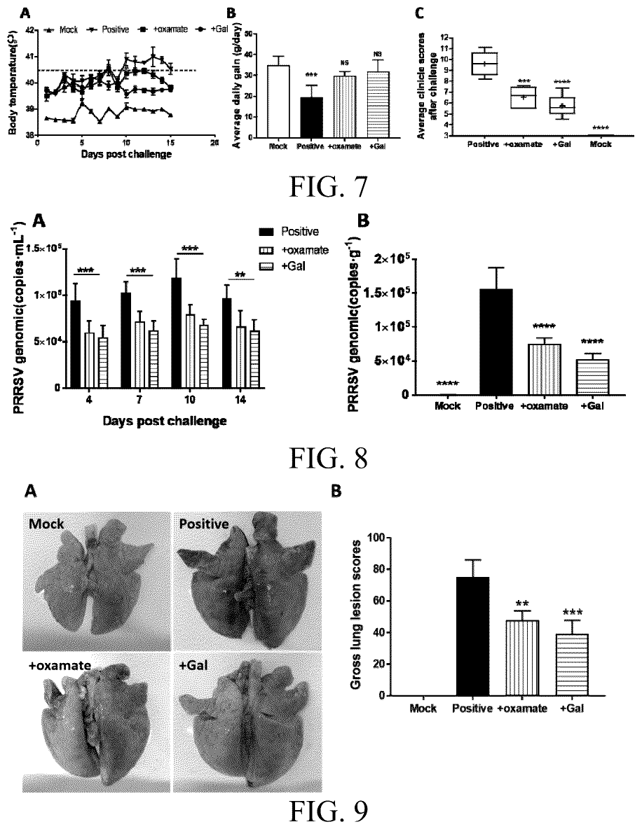

The rectal temperature (FIG. 7A), body weight (FIG. 7B) and clinical symptoms (FIG. 7C) of the piglets were measured daily 0-14 days after virus infection. The results showed that the piglets in the negative control group had no clinical fever, no obvious clinical symptoms, and a normal growth rate during the experiment. The piglets in the positive control group continued to have high fever (≥40.5° C.) on days 10-14 after virus infection, showed clinical symptoms such as loss of appetite, lethargy, rough hair, dyspnea, periocular edema and mild diarrhea, and had a weight growth rate significantly lower than that of the negative control group (P<0.001). However, the piglets in the sodium oxamate treatment group and the Galloflavin treatment group had a body temperature not exceeding 40.5° C., no obvious clinical symptoms, normal mental state and appetite, no respiratory symptoms such as dyspnea or cough, significantly relieved clinical symptoms compared with the positive control group (P<0.001), and a weight growth rate approximate to that of the negative control group (P>0.05).

### 2.3.2 Detection Results of Viremia and Viral Load of Lung Tissue

Serum was collected at the 1 dpi, 4 dpi, 7 dpi, 10 dpi, and 14 dpi, and the copy number of PRRSV genome cDNA was determined by qRT-PCR. The results showed that the virus content in the serum of the piglets in the sodium oxamate treatment group and the Galloflavin treatment group was significantly lower than that in the positive control group (FIG. 8A). The piglets were killed and dissected at 14 dpi, and the viral load in the lungs of each piglet was detected. The results showed that the PRRSV viral load in the lungs of the piglets in the two treatment groups was significantly lower than that in the positive control group (FIG. 8B) (P<0.0001).

### 2.3.3 Gross Lung Lesions of Piglets

The piglets were killed and dissected on day 14 after the challenge, and gross lung lesions of the piglets in each group were observed and scored statistically. The results showed that the lungs of the piglets in the negative control group were completely normal in appearance and had no obvious lesion; the lung tissue of the piglets in the positive control group had obvious consolidation and hemorrhage; and the piglets in the treatment groups had significantly minor lung lesions compared with the positive control group (FIG. 9A). The statistical results of the gross lung lesion scores showed that compared with the lung lesion scores of the piglets in the positive control group, the lung lesion scores of the piglets in the sodium oxamate treatment group was significantly lower (P<0.01), and the lung lesion scores in the Galloflavin treatment group were more significantly lower (FIG. 9B) (P<0.001).

### 2.3.4 Pathological Changes in Lungs of Piglets

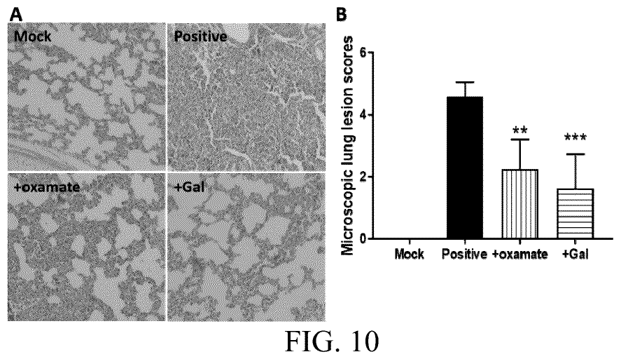

Histopathological changes of lungs were observed as follows: the lung tissue of piglets in the positive control group had obvious thickened alveolar septa, scattered hemorrhage in the alveolar septa, increased inflammatory cells and increased inflammatory exudates in the trachea; the lung lesions of the piglets in the sodium oxamate treatment group had mild thickened alveolar septa and inflammatory cell infiltration; and the lung lesions of the piglets in the Galloflavin treatment group were significantly alleviated, similar to those in the negative control group (FIG. 10A). Statistical results of microscopic lung lesion scores showed that the lung lesion scores of the negative control group and the treatment groups were significantly lower than those of the challenge control group (FIG. 10B) (P<0.01).

### 3. Discussion

PRRSV can infect pigs of any age, mainly causing reproductive disorders such as abortion, premature birth, stillbirth, weak fetus and mummified fetus in pregnant sows, and respiratory symptoms in piglets and fattening pigs. PRRSV was first reported in the United States in 1987 and has since spread rapidly around the world. American PRRSV (CH-1a strain) was first isolated from suspected PRRS cases in China in 1996 by Harbin Veterinary Research Institute of Chinese Academy of Agricultural Sciences. In 2006-2007, highly pathogenic HP-PRRSV swept across all provinces in China, and brought huge losses to the pig industry. The World Organization for Animal Health classifies PRRS as a Class B disease, and the Animal Epidemic Prevention Law of the People's Republic of China classifies classic PRRS as a Class II animal epidemic disease, and highly pathogenic PRRS as a Class I animal epidemic disease.

At present, in China, in addition to strengthened management, strict disinfection, etc., vaccine immunization is the main measure to prevent PRRS, using mainly inactivated vaccines and attenuated vaccines. However, due to the characteristics including easy mutation, multi-strain co-infection and persistent infection, vaccine immunization cannot completely control the spread of PRRSV. Moreover, so far there is no specific drugs for PRRSV. Therefore, development of a new and effective drug is crucial for the prevention and treatment of PRRS.

It is an effective treatment to find an effective way to inhibit virus replication, but it is complicated to screen out virus-inhibiting drugs in vitro and analyze their metabolic kinetics and toxic and side effects in vivo. Also, PRRSV has the characteristic of easy mutation, and the use of antiviral drugs may lead to emergence of drug-resistant strains. To break through the bottleneck, it is possible to analyze and explore treatment options from the perspective of the host. Metabolic immunology is a frontier research direction of modern immunology. Combining the host immune response with its metabolic changes can lead to discovery of drugs targeting the corresponding sites, thereby reducing damage to the host caused by viral infection.

This study links glycolysis to PRRSV replication by finding that GAPDH can promote PRRSV replication through its glycolytic activity. In normal mammalian cells, glucose is oxidized through glycolysis and enters the tricarboxylic acid cycle (TCA) to generate adenosine triphosphate (ATP) for intracellular life activities. This study found that the classical inhibitor of glycolytic pathway 2-DG has an inhibitory effect on PRRSV infection, confirming that the glycolysis process in host cells plays an important role in PRRSV replication. Also, this study found that lactate dehydrogenase inhibitors in the glycolysis process, sodium oxamate and Galloflavin, can both inhibit PRRSV replication. 2-DG is in high cost, and has a low cost performance in clinical application. Therefore, sodium oxamate and Galloflavin were used for treatment experiments in piglets. The results showed that the sodium oxamate and Galloflavin could alleviate clinical symptoms such as anorexia, fever (40-41° C.), lethargy, depression, and dyspnea caused by PRRSV, greatly reduce viremia and pulmonary viral load in infected piglets, and alleviate interstitial pneumonia caused by PRRSV, showing a great potential as a PRRSV therapeutic drug. This study found the PRRSV antagonists sodium oxamate and Galloflavin for the first time, and the sodium oxamate and Galloflavin have clear anti-PRRSV medicinal ingredients, are quality controllable, safe and non-toxic, and have a good application prospect in prevention and treatment of PRRSV.

1. A method for preventing or treating porcine reproductive and respiratory syndrome virus (PRRSV) infection comprising administering a drug comprising one or more inhibitors of glycolytic pathway to a subject in need thereof.

2. The method according to claim 1, wherein the inhibitors of glycolytic pathway are a hexokinase inhibitor and/or a lactate dehydrogenase inhibitor.

3. The method according to claim 1, wherein the hexokinase inhibitor is 2-deoxy-D-glucose; and the lactate dehydrogenase inhibitor is at least one of sodium oxamate and Galloflavin.

4. The method according to claim 1, wherein the drugs are prepared from the inhibitors of glycolytic pathway as an active ingredient.

5. The method according to claim 4, wherein the drug also contains a pharmaceutically acceptable carrier.

6. The method according to claim 4, wherein the dosage form of the drug is any one acceptable in veterinary pharmacy.

7. The method according to claim 2, wherein the drugs are prepared from the inhibitors of glycolytic pathway as an active ingredient.

8. The method according to claim 3, wherein the drugs are prepared from the inhibitors of glycolytic pathway as an active ingredient.

9. The method according to claim 7, wherein the drug also contains a pharmaceutically acceptable carrier.

10. The method according to claim 8, wherein the drug also contains a pharmaceutically acceptable carrier.

11. The method according to claim 7, wherein the dosage form of the drug is any one acceptable in veterinary pharmacy.

12. The method according to claim 8, wherein the dosage form of the drug is any one acceptable in veterinary pharmacy.
